(19) United States
(12) Patent Application Publication
(10) Pub. No.: US 2012/0169582 A1
(43) Pub. Date: Jul. 5, 2012

(54) SYSTEM READY SWITCH FOR EYE TRACKING HUMAN MACHINE INTERACTION CONTROL SYSTEM

(75) Inventor: Michael Dean Tschirhart, Ann Arbor, MI (US)

(73) Assignee: VISTEON GLOBAL TECHNOLOGIES, VAN BUREN TOWNSHIP, MI (US)

(21) Appl. No.: 12/984,968

(22) Filed: Jan. 5, 2011

(51) Int. Cl. G06F 3/01; A61B 3/113

(52) U.S. Cl. 345/156; 351/209

(57) ABSTRACT

The invention relates to a system and method for activating a visual control interface, and in particular, for activating a visual control interface using an eye tracking system in a vehicle. A switch is used to activate and deactivate a control section of an eye tracking system in a human-machine interaction control system. The system allows a driver (or operator) of a vehicle to signal the system with selection of the switch to activate or deactivate the control section, thereby providing functional support to the driver when desired, but remaining inconspicuous otherwise.

### BACKGROUND OF THE INVENTION

1. Technical Field

The invention relates to a system and method of activating a visual control interface, and in particular, to activating a visual control interface using an eye tracking system in a vehicle.

2. Discussion

Eye tracking technology systems are primarily used for driver gaze status detection and alerts. For example, detecting a driver's gaze which deviates from a roadway, in which, after a sufficient length of time, the system may issue an alert to the driver that an unsafe driving condition exists, i.e. namely failing to look at the road.

As illustrated in FIG. 1 of U.S. Pat. No. 6,926,429, the contents of which are hereby incorporated by reference, an apparatus for eye tracking includes conventional elements of a head up display (HUD) system along with an IR sensor/camera 50 providing an input to a processing platform 52, a ring of IR illuminators 54 disposed near the IR sensor/camera 50, and an IR mirror 56 that reflects radiation from the IR illuminators onto aspheric mirror 40. Aspheric mirror 40, in turn reflects the radiation onto windshield 42 which may have an IR reflection enhanced surface 58. The radiation reflects off surface 58 and showers an eye 60 and the face (not shown on FIG. 1) of the driver. The resulting image of the eye and face of the driver is reflected in sequence off surface 58, aspheric mirror 40, and IR mirror 56 and is received by IR sensor/camera 50 The signal from sensor/camera 50 is passed to processing and control circuitry in processing platform 52 and used in the manner described below, the processing and control circuitry also controlling the other elements of the system IR sensor/camera 50 could include electronic pan tilt to compensate for head and eye movement Illumination sources other than IR may be provided, as long as the other illumination sources are non-intrusive. The system 10 has no moving parts.

Recent developments in eye tracking technology enable systems to use eye tracking for control of user interfaces. For example, eye tracking may be used to deter mine an eye gaze at an audio preset, which preset can be activated through a variety of control mechanisms. However, current eye tracking systems for control systems can be distracting and irritating to drivers and can often misread the intended “gaze” of the driver, as they continuously monitor the eyes of a driver (i.e. they are always on). These conventional systems in vehicle applications are overwhelmingly focused on driver monitoring. Algorithms in these systems tend to focus on assessing driver gaze and infer various aspects of the driver's condition. Systems then infer behavior and make adjustments and/or provide alerts.

U.S. Publication 2006/0259206, the contents of which are hereby incorporated by reference, discloses a vehicle operator monitoring system in which a series of ocular profiles of a vehicle operator are captured. An analysis of the profiles is conducted of the vehicle operator, and the analysis is applied to a vehicle system to maintain or adjust a state of the vehicle system. With reference to FIG. 2, a passenger compartment 12 of a vehicle 10 is shown equipped with a vehicle operator attentiveness imaging system having a video imaging camera to carry out the monitoring and capturing of the series of ocular profiles, as well as applying the analysis of the captured data to the vehicle system.

### SUMMARY OF THE INVENTION

The invention relates to a system and method of activating a visual control interface, and in particular, to activating a visual control interface using an eye tracking system in a vehicle.

In one embodiment of the invention, a visual control interface in a vehicle, including an eye tracking system for tracking eye movement of an operator of the vehicle; and an input for activating and deactivating at least one section of the eye tracking system.

In another embodiment of the invention, a method for visual control of an interface in a vehicle, includes tracking eye movement, with an eye tracking system, of an operator of the vehicle; and activating/deactivating at least one section of the eye tracking system using an input.

In one aspect of the invention, the eye tracking system comprises a monitoring section and a control section.

In another aspect of the invention, the visual control interface includes a database storing data corresponding to eye movement tracked by the eye tracking system; a network interface configured to actively connect to a network and the database; and a processor for processing the data tracked by the eye tracking system and stored in the database.

In still another aspect of the invention, the monitoring section monitors the operator's eye to acquire information associated with eye movement; and the control section, when activated, allows the operator of the vehicle to select an item on the visual control interface based on the acquired eye movement, and when in the deactivated state, prevents the operator of the vehicle from selecting an item on the visual control interface.

In yet another aspect of the invention, selection of the input activates/deactivates the control section, and the monitoring section continuously monitors the eye of the operator in the activated and deactivated state of the control section.

In another aspect of the invention, the control section is activated/deactivated automatically as determined by a preset rule.

In yet another aspect of the invention, items appearing on the visual control interface correspond to at least one of a device and function of the vehicle, and the visual control interface is at least one of a heads up display, navigation display, television display, dash board display, instrument panel display, mirror display and monitor.

In still another aspect of the invention, the input includes or has a corresponding indicator to indicate one of the activated or deactivated states.

In another aspect of the invention, the input is at least one of a switch, button and voice control.

In still another aspect of the invention, the network is at least one of an internet, intranet, WAN, LAN, telecommunications network and world wide web.

### BRIEF DESCRIPTION OF THE DRAWINGS

The present invention will become more fully understood from the detailed description given here below, the appended claims, and the accompanying drawings in which:

FIG. 1. illustrates an eye tracking system in accordance with the prior art.

FIG. 2 illustrates a vehicle operator being monitored by an eye tracking system in accordance with the prior art.

FIG. 3 illustrates an exemplary “system ready” switch in accordance with an embodiment of the invention.

FIG. 4 illustrates an exemplary selection of items on a control interface using eye tracking in accordance with one embodiment of the invention.

FIG. 5 illustrates an exemplary block diagram of a system in accordance with one embodiment of the invention.

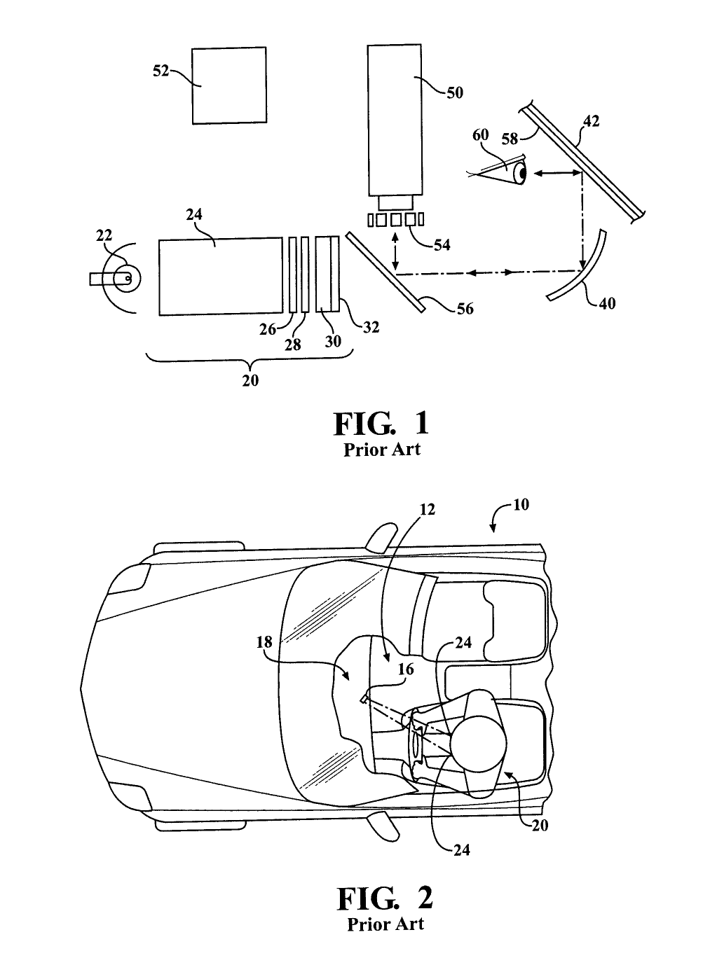

### DETAILED DESCRIPTION OF THE PREFERRED EMBODIMENT

A system and method is provided for activating a visual control interface, and in particular, for activating a visual control interface using an eye tracking system in a vehicle.

A switch (e.g. “system ready” switch) is used to activate and deactivate a control section of an eye tracking system in a human-machine interaction control system. The system 1 allows a driver (or operator) of a vehicle 10 to signal the system 1 to activate or deactivate the control section, thereby providing functional support to the driver when desired, but remaining inconspicuous otherwise. Switch 100 may be any input, including for example a solid state or mechanical switch, voice control, or other methods. Moreover, the invention is not limited specifically to a switch per se, but may be a button or any interface or input capable of providing on/off functionality within the context of the invention. As explained below with reference to the Figures in more detail, when an driver of a vehicle seeks to use eye tracking for control of a control interface, a switch 118 in the vehicle is selected to activate the system, thereby and activating the gaze detection and control mechanisms. Once the driver completes the control activation sequence (e.g. has completed using the eye tracking to control the control interface), another signal (or removal of the first signal) restores the eye tracking for the control section to an unobtrusive “deactivated” state. It is appreciated that the eye tracking technology employed in the invention can be any eye tracking technology readily understood by the skilled artisan and as known in the art. Similarly, use of such data acquired during eye tracking and gazing may be applied using any known techniques in the art.

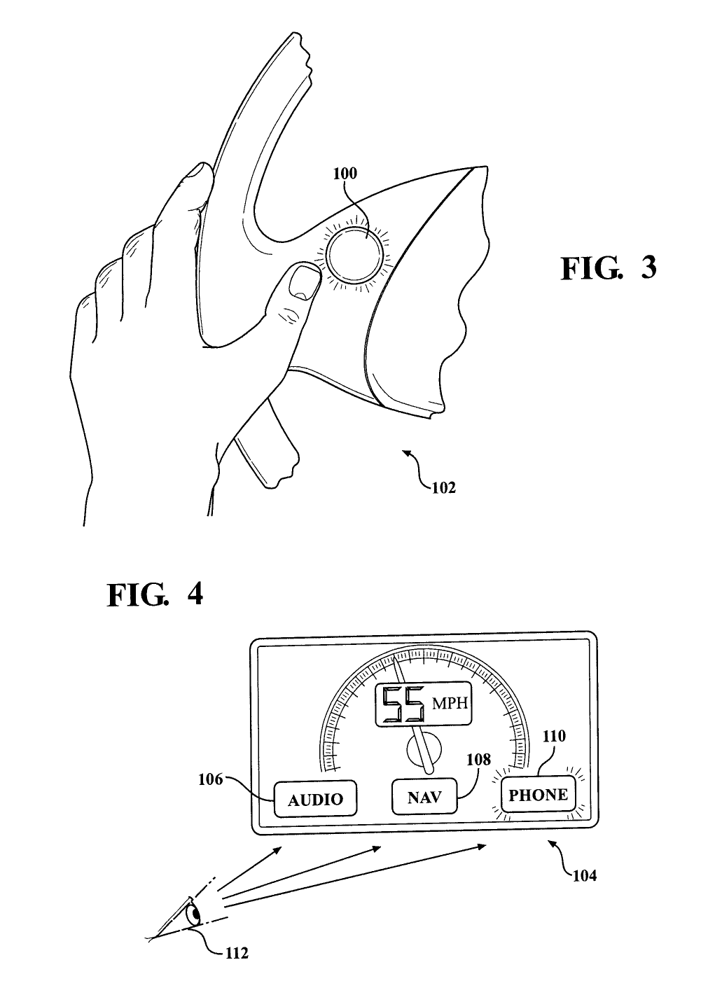

FIG. 3 illustrates an exemplary “system ready” switch in accordance with an embodiment of the invention. According to one embodiment of the invention, a driver (or operator) of the vehicle 10 may activate/deactivate the control section of eye tracking system 116 by selection of a “system ready” switch 100 located, for example as depicted, on the steering wheel 102 of vehicle 10. It is appreciated, however, that the invention is not limited to this specific embodiment and any method known to the skilled artisan may be used in order to activate/deactivate the control section of the eye tracking system. For example, instead of a switch, the ability for the system to enable the control section of the eye tracking system, such that a driver controls the interface, may be activated/deactivated by a voice command, selection of a button located anywhere in the vehicle, such as on the dash board or on the control interface (touch screen or otherwise), automatically activated/deactivate based on a rule or set of rules (for example, speed of the vehicle, time of day, etc.), or for example by a particular sequence or movement of the drivers eyes which indicate activation or deactivation of the eye tracking system.

FIG. 4 illustrates an exemplary selection of items on a control interface using eye tracking in accordance with one embodiment of the invention. In the activated state, the driver of the vehicle (or a passenger in the vehicle) can control a visual control interface 104 in the vehicle using his/her eyes 112. The visual control interface 104, in the depicted embodiment, shows three selectable items, namely audio 106, nav 108 and phone 110. These items are exemplary in nature, and it is appreciated that the visual control interface is not limited to such an embodiment. Rather, the eye tracking system of the invention may be used to operate any control interface in which a driver may view such interface, including interfaces on the dash board, heads up displays, optical images on mirrors and the like. Specifically, the eye tracking system 116 of the vehicle allows the driver to control each of the selectable items 106, 108, 110 displayed on visual control interface 104. For example, when a driver focuses his eyes 112 on an item appearing on the display (interface), the eye tracking system will cause the item to be highlighted and/or selected thereby enabling the device or function associated with the displayed item. Once the selection has been completed, the driver may place the control section of the eye tracking system back into “deactivated” mode. Use of this system enables the driver to continue using both hands while driving without adding unnecessary driver distraction. Moreover, since the control section can be deactivated, the eye tracking system will not misinterpret or otherwise active an item on the visual control interface accidentally. At the same time, however, the eye tracking system continues to monitor the drivers eye and provide feedback to the system 1 in a manner readily understood to the skilled artisan.

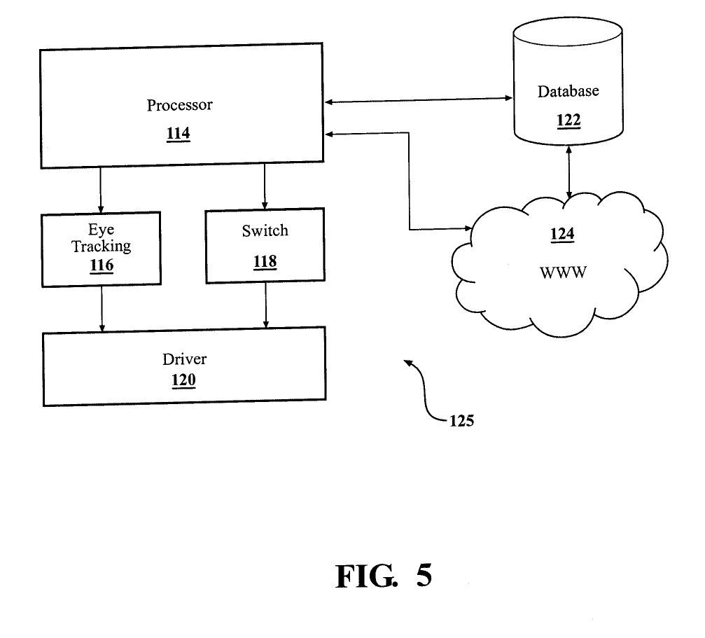

FIG. 5 illustrates an exemplary block diagram of a system in accordance with one embodiment of the invention. The system 125 includes, for example, processor 114, eye tracking system 116, switch 118, driver 120, database 122 and world wide web (or any other type of network) 124. As explained above, eye tracking system 116, which includes at least a monitoring section and a control section, may be any system used in the art and readily understood to the skilled artisan. Processor 114 may be any processor as readily understood by the skilled artisan. Database 122 stores information acquired during monitoring of the driver eyes by the eye tracking system 116, as well as any other information usable by processor 114 to evaluate, monitor and determine outcomes and events based on the such data and information. Alternatively, or additionally, data may be accessed and provided via the world wide web 124 or any other network connected to database 122 and processor 114. Moreover, the database 122 and network connections may be located in or outside of the vehicle, and accessible either by wire or wirelessly.

In operation, the eye tracking system 116 may be used as follows. Notably, the eye tracking system 116 in one embodiment of the invention is divided into two separate functions-monitoring and control. The eye tracking system 116 continuously monitors the drivers eyes 112 to relay information to processor 114, which acts upon the monitored information accordingly. The control function of the eye tracking system 116, on the other hand, may be operatively selected and deselected by the driver in order to allow or disallow functionality of the control functionality. More specifically, upon entry into the vehicle, the system 1 is activated (or remains in the deactivated state until activated otherwise). Activation of system 1 includes activation of the monitoring and control sections of eye tracking system 116. Should the driver desire to deactivate (or activate) the control section of the eye tracking system 116, he/she may select switch 118. Selection of switch 118 that changes the control section from an activated to a deactivated state places the control section in an “off” state such that monitoring of the driver's eyes continues, but the ability of the driver to operatively select items on visual control interface 104 is no longer achievable. On the other hand, selection of switch 118 that changes the control section from a deactivated to an activated state places the control section in an “on” state such that monitoring of the driver's eyes continues and the control section is enabled, thereby allowing the driver to actively select items appearing on the visual control interface 104 using his/her eyes.

The foregoing invention has been described in accordance with the relevant legal standards, thus the description is exemplary rather than limiting in nature. Variations and modifications to the disclosed embodiment may become apparent to those skilled in the art and do come within the scope of the invention. Accordingly, the scope of legal protection afforded this invention can only be determined by studying the following claims.

What is claimed is:

1. A visual control interface in a vehicle, comprising:
an eye tracking system for tracking eye movement of an operator of the vehicle; and
an input for activating and deactivating at least one section of the eye tracking system.

2. The visual control interface of claim 1, wherein the eye tracking system comprises a monitoring section and a control section.

3. The visual control interface of claim 1, further comprising:
a database storing data corresponding to eye movement tracked by the eye tracking system;
a network interface configured to actively connect to a network and the database; and
a processor for processing the data tracked by the eye tracking system and stored in the database.

4. The visual control interface of claim 2, wherein
the monitoring section monitors the operator's eye to acquire information associated with eye movement; and
the control section, when activated, allows the operator of the vehicle to select an item on the visual control interface based on the acquired eye movement, and when in the deactivated state, prevents the operator of the vehicle from selecting an item on the visual control interface.

5. The visual control interface of claim 4, wherein selection of the input activates/deactivates the control section, and the monitoring section continuously monitors the eye of the operator in the activated and deactivated state of the control section.

6. The visual control interface of claim 4, wherein the control section is activated/deactivated automatically as determined by a preset rule.

7. The visual control interface of claim 4, wherein
items appearing on the visual control interface correspond to at least one of a device and function of the vehicle, and
the visual control interface is at least one of a heads up display, navigation display, television display, dash board display, instrument panel display, mirror display and monitor.

8. The visual control interface of claim 2, wherein the input includes or has a corresponding indicator to indicate one of the activated or deactivated states.

9. The visual control interface of claim 2, wherein the input is at least one of a switch, button and voice control.

10. The visual control interface of claim 3, wherein the network is at least one of an internet, intranet, WAN, LAN, telecommunications network and world wide web.

11. A method for visual control of an interface in a vehicle, comprising:
tracking eye movement, with an eye tracking system, of an operator of the vehicle; and
activating/deactivating at least one section of the eye tracking system using an input.

12. The method of claim 11, wherein the eye tracking system includes a monitoring section and a control section.

13. The method of claim 11, further comprising:
storing, in a database, data corresponding to eye movement tracked by the eye tracking system;
actively connecting to a network and the database using a network interface; and
processing the data tracked by the eye tracking system and stored in the database.

14. The method of claim 12, wherein
the monitoring section monitors the operator's eye to acquire information associated with eye movement; and
the control section, when activated, allows the operator of the vehicle to select an item on the visual control interface based on the acquired eye movement, and when in the deactivated state, prevents the operator of the vehicle from selecting an item on the visual control interface.

15. The method of claim 14, wherein selection of the input activates/deactivates the control section, and the monitoring section continuously monitors the eye of the operator in the activated and deactivated state of the control section.

16. The method of claim 14, wherein the control section is activated/deactivated automatically as determined by a preset rule.

17. The method of claim 14, wherein
items appearing on the visual control interface correspond to at least one of a device and function of the vehicle, and
the visual control interface is at least one of a heads up display, navigation display, television display, dash board display, instrument panel display, mirror display and monitor.

18. The method of claim 12, wherein the input includes or has a corresponding indicator to indicate one of the activated or deactivated states.

19. The method of claim 12, wherein the input is at least one of a switch, button and voice control.

20. The method of claim 13, wherein the network is at least one of an internet, intranet, WAN, LAN, telecommunications network and world wide web.
